(19) United States
(12) Patent Application Publication
(10) Pub. No.: US 2019/0099240 A1
(43) Pub. Date: Apr. 4, 2019

(54) ROTARY ENDODONTIC FILE IN BASKET FORM

(71) Applicant: DZEAL PRIVATE LIMITED, Navi Mumbai (IN)

(72) Inventor: LAVKESH BANSAL, Navi Mumbai (IN)

(21) Appl. No.: 16/094,588

(22) PCT Filed: Jun. 23, 2017
(86) PCT No.: PCT/IN2017/050256

§ 371 Date: Oct. 18, 2018

(30) Foreign Application Priority Data
Jun. 26, 2016 (IN) 201621014443

(51) Int. Cl. A61C 5/48; A61C 5/42

(52) U.S. Cl. CPC A61C 5/48; A61L 31/022; A61C 2201/007; A61C 5/42

(57) ABSTRACT

Present invention provides a rotary endodontic file for root canal treatment of tooth with particular arrangement of wires so as to form a basket shape. The file is flexible and has increased resistance to cyclic fatigue breakage. Said rotary endodontic file is flexible, easy in operation, simple in design and it reduces 5 torsional as well as cyclic failure. Also, said file can be operated by using available endo-motors.

### FIELD OF INVENTION

The present invention relates to a medical device used in dentistry.

More particularly the invention relates to a rotary endodontic file used in the treatment of root canal.

Still more particularly the invention provides a novel fluteless rotary endodontic file for endodontic treatments which reduces the chances of cyclic failure seen in root canal treatments.

### BACKGROUND OF INVENTION

Present invention is described herein below with reference to a Rotary endodontic file in basket form; however any description and drawings herein after should not be construed to limit the scope of present invention in any manner.

Endodontic therapy or root canal therapy is a sequence of treatment for the infected pulp of a tooth which results in the elimination of infection and protection of the decontaminated tooth from future microbial invasion. Root canals and their associated pulp chamber are the physical hollows within a tooth that are naturally inhabited by nerve tissue, blood vessels and their cellular entities. Endodontic therapy involves the removal of these structures, the subsequent shaping, cleaning, and decontamination of the hollows with small files and irrigating solutions, and the filling of the decontaminated canals.

Rotary endodontic file is an engine-driven tapered and pointed endodontic instrument made of nickel titanium alloy with cutting edges used to mechanically shape and prepare the root canals during endodontic therapy or to remove the root canal obturating material while performing retreatment. Root canal is done with either manually, using a series of stainless steel and/or nitinol hand-held files, and/or mechanically, using rotary nitinol files in rotational motion, and/or mechanically, using ultrasonic stainless steel or nitinol file in vibrating motion, adapted to fit a standard or dedicated dental handpiece.

The manual process is slow, time consuming and tiring to perform. It is unavoidable and necessary to prepare the canal manually up to certain size before starting with rotary or ultrasonic files.

The mechanical process using rotary endodontic file has improved the speed and efficiency of cleaning the root canals substantially. Since these are used at 200-1500 Revolutions per minute, there is a great chance of cyclic or torsional failure/fracture of instrument in the canal. A number of systems dedicated machines with full control on speed, torque and combination of clockwise and anti-clockwise rotation has been developed to prevent these failures. Different designs and material processing has been developed to prevent these failures.

The mechanical process using sonic or ultrasonic file has limited application as these need free space around the instrument for optimal performance and that is not always possible in thin root canals.

Research has shown that mechanical process is surely better than the manual process of performing the root canal treatment. But the few shortcomings in this approach are:
a. In canal breakage of files. Broken files are difficult to remove and they need additional instruments and equipment. Bypassing or removing the broken instrument in the canal compromise the quality of services rendered in terms of biomechanical preparation and/or remaining strength of tooth.
b. Torsional fracture is mainly of thinner files due to the engagement of flutes of file in to the wall of canal or due to intentional or inadvertent push of file tip in to the narrow canal.
c. Cyclic fracture is mainly of thicker files due to the repeated compression and flexion of file at the curves of canal.
d. Round cross section of files does not adequately clean the oblique or ribbon shaped canals or do so at the expense of removing unnecessary dentine from walls. It causes perforation of the wall or weakening of tooth root.
e. There is significant increase in the cost of different file systems and related instruments.
f. Handling different equipment and multiple file system is demanding in terms of expertise required to handle the company specified operatory manual.

Attempts have been made in the prior arts in order to overcome above mentioned problems by providing new and efficient designs of rotary endodontic files.

U.S. Pat. No. 8,790,116, titled “Endodontic file” discloses an endodontic file for rotary hand piece. It comprises a coupler element for coupling to the handpiece, a central longitudinal flexible core including a central flexible cable supported by the coupler element, and a helical wire surrounding the core.

It also discloses a method for preparation of root canal of a tooth during an endodontic treatment using said endodontic file.

The design of endodontic file described in U.S. Pat. No. 8,790,116 may help in decreasing the chance torsional failure of file, but at the same time it increases the chance of extensive preparation at the point where outer helical wire halts and the inner core extend beyond to prepare the apical portion.

A PCT application no WO2008102352, discloses an endodontic file with a cord and wire wound around, a major portion of the cord and having a binder containing abrasive particles on outer surface. Said cord is having plurality of twisted threads.

The file according to the present invention is inserted by imparting a rotational movement unlike files in prior arts inserted into a root canal by pushing the file axially into the root canal. Owing to the substantial flexibility of the file according to the present invention, the file is effective in cleaning convoluted root canals since it merely closely follows the canal's contours without injuring the dentine layer.

PCT application WO2008102352, provides a new endodontic file which may decrease the chance of torsional failure but the abrasive particle present on outer surface of the file has every chance of getting separated and lodged in to the fine crevices of root canal.

United States Patent application No US2005026109, discloses a series of multi-tapered endodontic files used in root canal treatment. Said files have tapered flute portion. The files have 2 portions with different tapers. The files are provided in sets of at least four with the tapers of the first portions of all of the files decreasing incrementally from the first to the last file of the set.

The endodontic file described in US2005026109, address the issue of different curvature at different level of root canal that are round in cross section, but working in oval files will have same disadvantage like traditional files that it either does not reach the smaller ends or over prepared the canal to reach every portion.

Hence, there is a need of rotary endodontic file which is flexible, easy in operation, simple in design and which reduces torsional as well as cyclic failure.

Also, there is a need of endodontic file which can operate by using available endo-motors.

### OBJECTS OF THE INVENTION

The main object of present invention is to provide a rotary endodontic file for root canal treatment of tooth.

Another object of the present invention is to provide a novel structure of rotary endodontic file for reducing torsional as well as cyclic failures.

### SUMMARY OF THE INVENTION

Present invention provides a rotary endodontic file for root canal treatment of tooth with particular arrangement of wires so as to form a basket shape.

The file is flexible and has increased resistance to cyclic fatigue breakage. A rotary endodontic file of a present invention is in basket form wherein wires are arranged in such a way that they meet together at lower portion called Edge portion and Connecting section at the upper portion of the endodontic file and thus form a basket of wires.

A fluteless rotary endodontic file for cleaning and extirpating the walls of a root canal comprising; an Edge portion which act as a single point of contact of wires protruding from lower end of Connecting section at the lower portion of the said file; a Working section further comprising atleast two thin, flexible wires arranged in the form of basket attached to Edge portion at lower end and Connecting section at upper end of the said file; a Connecting section for connecting Working section to Coupling section and a Coupling section acting as a handle for said endodontic file for holding the device during root canal treatment.

The invention further relates to variations of said rotary endodontic file in length, diameter and arrangement of flexible wires so as to form a basket shape.

### BRIEF DESCRIPTION OF THE DRAWINGS

FIG. 1 illustrates a schematic representation of the rotary endodontic file of present invention with all its components

FIG. 2 relates to a front view as well as right side view of variation I of rotary endodontic file of present invention

FIG. 3 relates to a front view as well as right side view of variation II of rotary endodontic file of present invention

FIG. 4 relates to a front view as well as right side view of variation III of rotary endodontic file of present invention

FIG. 5 relates to a front view as well as right side view of variation IV of rotary endodontic file of present invention

TABLE NO. 1
legends used in the drawings and their description

| Legend | Name of the part |
|---|---|
| D0 | Tip of the rotary endodontic file |
| D1 | The point where working section ends |
| D2 | The point where there is maximum flaring |
| | (max. diameter) of basket of wires |
| D3 | The point where the working section starts |
| D4 | The point where the connecting section starts |
| D5 | Uppermost end of the rotary endodontic file |

### DETAILED DESCRIPTION OF THE DRAWINGS

The present invention is illustrated by way of example, and not by way of limitation, in the figures of the accompanying drawings and in which like reference numerals refer to similar elements.

The present invention is better understood by reference to the detailed figures and description set forth herein.

Present invention provides a rotary endodontic file wherein thin and flexible files are arranged in such a manner that they form a basket shape.

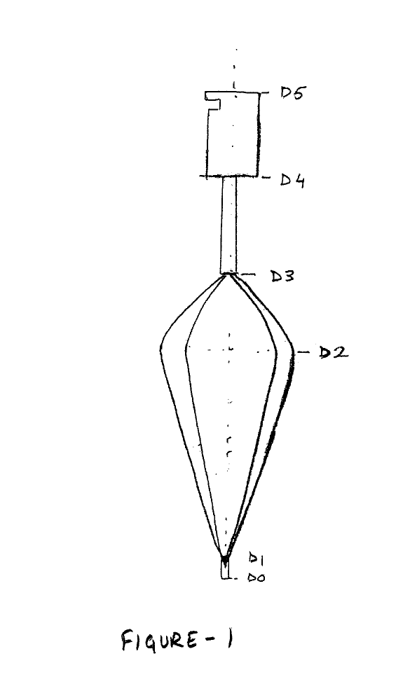

Referring to FIG. 1, the rotary endodontic file of present invention with all its components is illustrated.

Said rotary endodontic file comprising; a tip (D0) which is the lowest end of the file, an Edge portion (D0-D1) acting as a single point of contact for plurality of wires protruding from (D3) where the working section starts; a Working section (D1-D3) comprising atleast two wires; a Connecting section (D3-D4) for connecting working section (D1-D3) to coupling section (D4-D5) and a Coupling section (D4-D5) or handle for holding the device during the dental treatment.

Said rotary endodontic file wherein; said Edge portion (D0-D1) is in range of 0.5 mm to 7 mm in length and may have cylindrical shape or any other polygonal shape with diameter in the range of 0.05 mm-0.8 mm and may have flutes or cutting edges. Preferably the tip is round, non cutting and not engaging.

Said Working section (D1-D3) comprises of atleast two thin, flexible flared wires and has gradual increase of diameter from point D1 till the point of maximum flaring diameter D2 and then gradual decrease of diameter till the wires joined together at a point D3. Said wires are arranged in such a way that collectively they form a basket shape. The wires are attached to Edge portion at point D1 and Connecting section at D3.

In one preferred embodiment, said working section (D1-D3) may have four thin, flexible wires having thickness in the range of 0.02 mm to 0.4 mm.

In one embodiment, each file may have wires of same or different thickness, shape, cross section, length or material. Different thickness or shapes may need depending upon the acuteness of curvature normally found in root canals of teeth. Said wires may have same thickness throughout its length or it may differ at different sections of basket. Each wire is either straight or tapered from 1% to 16%. Thicker wire may give more stiffness which will be helpful in wider canal of anterior teeth and thinner wires will be ideal for narrow canal of posterior teeth.

Said wires may arranged to form a single basket from top to bottom or may arranged to form two or more baskets with different shape, size, depth, material, spatial plane arrangement respective to each other. Different arrangement will give different cutting potential to wire.

Said wires are made from the material selected from stainless steel, steel alloy, high temperature shape memory alloys, any super elastic material such as but not limited to, nitinol, nitinol-niobium alloy, nitinol alloys with zirconium, any nitinol alloy with or without trace elements, tantalum alloys with or without trace elements, titanium, beta-titanium, titanium alloys with zirconium, any titanium alloy with or without trace elements, shape memory ceramics, silicones or any other polymer.

In another embodiment, the straight distance between (D1-D3) is in the range of 12 mm-21 mm and the angle between wires and long axis, formed at the edge side (at D1) that gives the basket shape may range from 1 degree to 24 degree. The increase in flaring due to increase in angle will help in using different file in each part of root canal e.g. an angle of 24% on a shorter file may used for orifice widening. File with smaller angles may used progressively as the width of root canal narrows.

In yet another embodiment, the distance between point D2 that is maximum diameter and D1 is in the range of 5 mm-14 mm and linear distance between D2 to D3 is in the range of 1 mm-10 mm.

Said Connecting section (D3-D4), is a solid or hollow rod that connects the Working section (D1-D3) to the Coupling section of file. The thickness of this Connecting section ranges from 0.2 mm-1 mm.

Said Coupling section (D4-D5), is designed as per the most common available engine driven friction grip or latch grip handpiece as well as depending upon need for specific movements. Else a customised hand-piece or motor or equipment for its use may be made separately. Said coupling section acts as a handle for the rotary endodontic file of present invention.

This part may be in shape of standard ISO specified design for manual endodontic files, to make the file to be used manually.

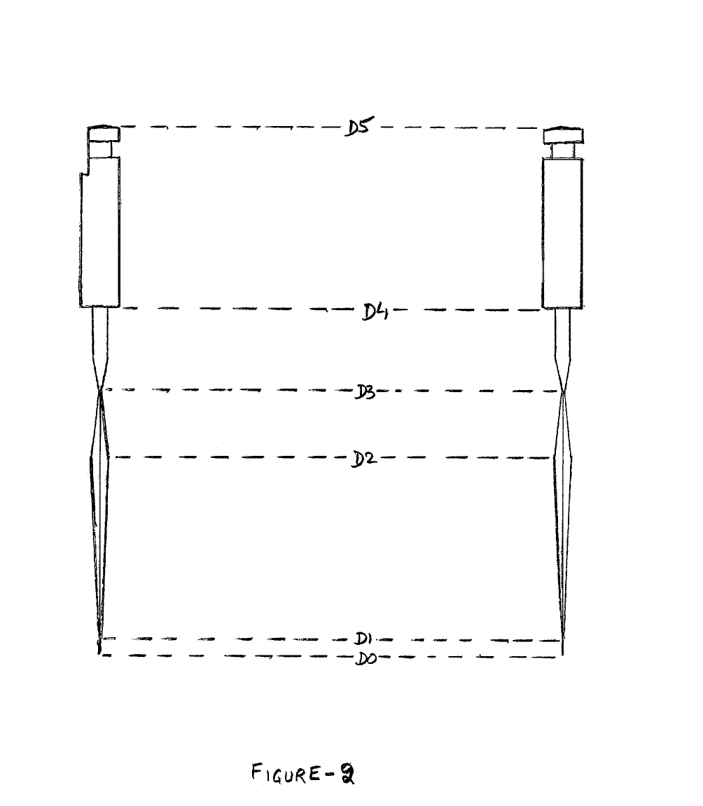

Referring to FIG. 2, a front view as well as right side view of variation I of rotary endodontic file of present invention is illustrated.

Variation I comprise of atleast two thin, flexible flared wires joined in cross hair arrangement at tip (D0) and have gradual increase of diameter from point D1 till the point of maximum flaring diameter D2 and then gradual decrease of diameter till the wires joined together at a point D3. Said maximum diameter (D2) is in the range of 0.4 mm to 6 mm. The distance D2-D3 is in the range of 1 mm-10 mm to meet at axis in line with tip (D0). The Connecting section (D3-D4) wherein wires run together is in range of 5 mm to 15 mm in length and then attached to Coupling section (D4-D5).

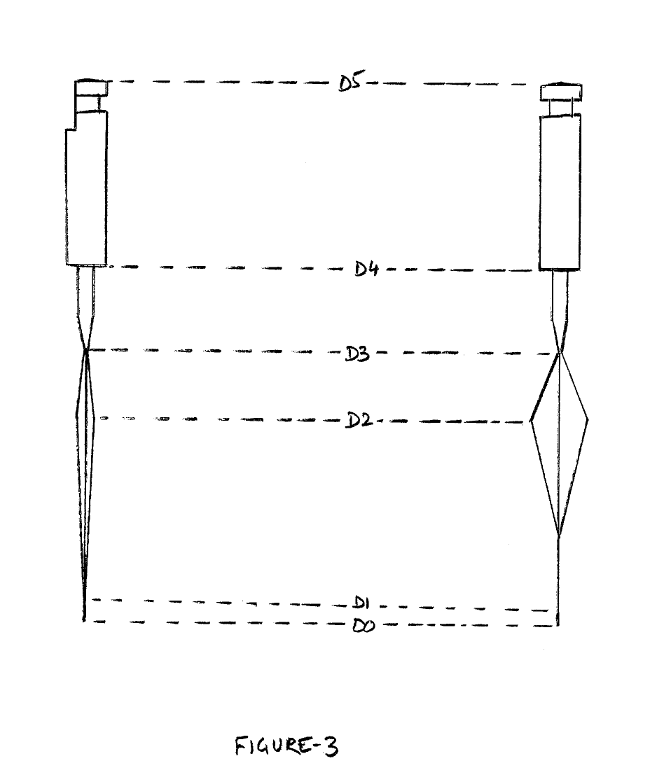

Referring to FIG. 3, a front view as well as right side view of variation II of rotary endodontic file of present invention is illustrated.

Variation II comprise of two baskets of wires arranged one above the other wherein; first basket comprises of atleast two thin, flexible flared wires joined together in the same plane at tip (D0) forming Edge portion (D0-D1) of 1 mm and then has gradual increase in diameter from point D1 till the point of maximum flaring diameter D2 and then gradual decrease of diameter till the wires joined together at a point D3. Said maximum flaring diameter for first basket (D2) is in the range of 0.4 mm to 6 mm. The second basket arranged in a perpendicular plane with respect to first basket at a distance of 6 mm from tip of first basket. Said second basket comprises of atleast two thin, flexible flared wires joined at tip (D0) forming Edge portion (D0-D1) of 6 mm and then flare at an angle twice of the first basket. The maximum flaring diameter for second basket is 6 mm. The wires of second basket converge at a point D3 and length of Connecting section (D3-D4) is 4 mm. Tips of both the baskets are joined together with another single wire of 6 mm. The length of Connecting section (D3-D4) is 5 mm that connects the converging point of baskets to Coupling section (D4-D5).

This design may helpful in curved canal, where three wires at the tip easily follow the path of curvature and the double flaring of second basket will start working as orifice widener in the root canal progress. This widening of orifice will help to reduce the strain in the wires of first basket.

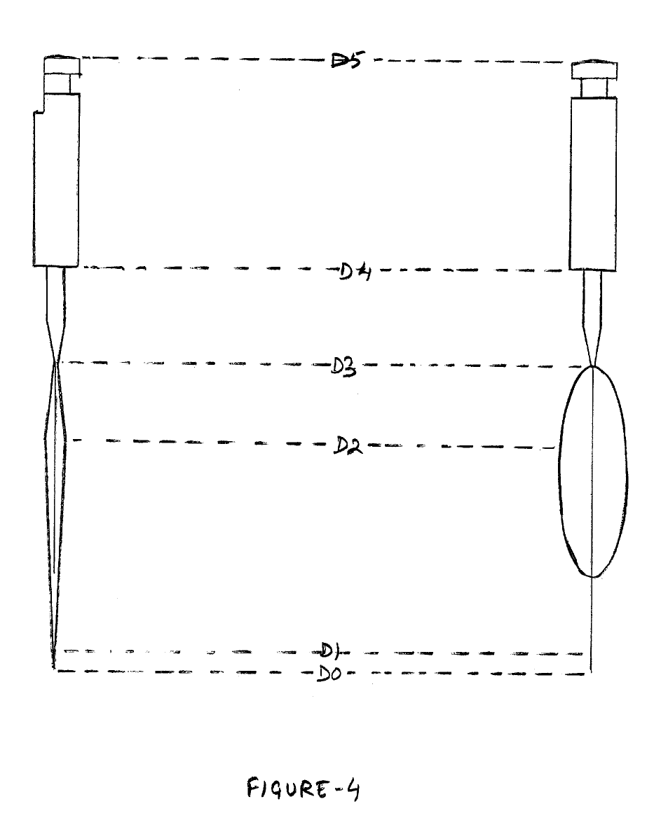

Referring to FIG. 4, a front view as well as right side view of variation III of rotary endodontic file of present invention is illustrated.

Variation III comprise of two baskets of wires wherein; first basket comprises of atleast two thin, flexible flared wires joined together in the same plane at tip (D0) forming Edge portion (D0-D1) of 1 mm and then has gradual increase in diameter from point D1 till the point of maximum flaring diameter D2 and then gradual decrease of diameter till the wires joined together at a point D3. Said maximum flaring diameter for first basket (D2) is in the range of 0.4 mm to 6 mm. The second basket arranged in a perpendicular plane with respect to first basket at a distance of 6 mm from tip of first basket. Said second basket comprises of a single wire in a loop shape. Two baskets converge at a point D3. The Connecting section (D3-D4) is of 5 mm in length means for connecting converging point (D3) to Coupling section or handle (D4-D5).

This design may helpful in thin curved canal, where two wires of first basket easily follow the path of curvature and the loop part of second basket start working as orifice widener as one progress in the root canal. This widening of orifice may help to reduce the strain in the wires of first basket.

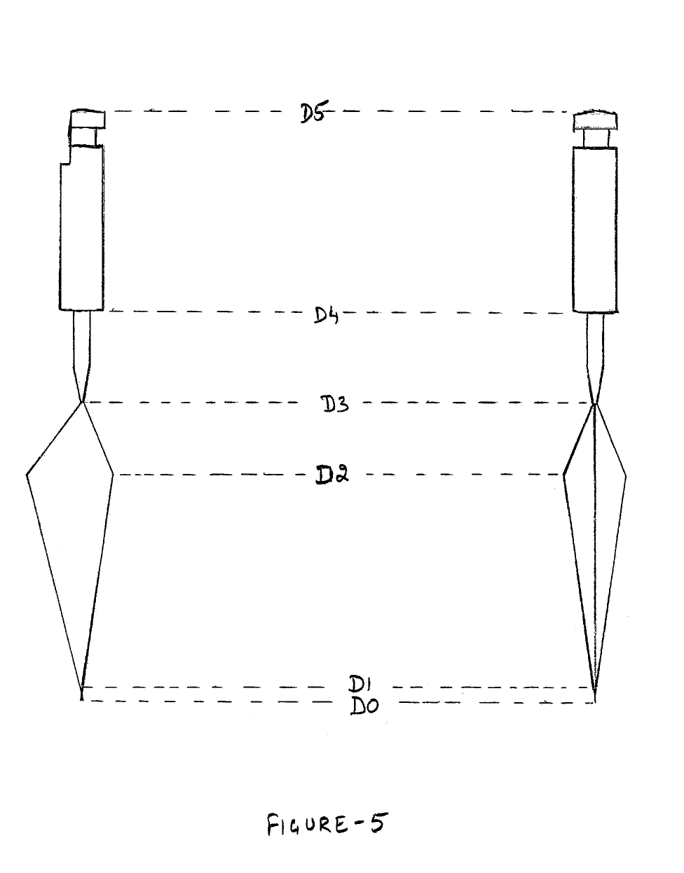

Referring to FIG. 5, a front view as well as right side view of variation IV of rotary endodontic file of present invention is illustrated.

Variation IV comprises of atleast two thin, flexible flared wires arranged in a basket shape form. Said wires joined together at tip (D0) converge at a tip (D0) forming Edge portion (D0-D1) of 1 mm and then has gradual increase in diameter with a constant angel from point D1 till the point of maximum flaring diameter D2 and then gradual decrease of diameter till the wires converge at a point D3. Said maximum flaring diameter for basket (D2) is in the range of 0.4 mm to 6 mm. The distance between D1-D3 is in the range of 12 mm to 21 mm to meet at axis in line with tip (D0). The Connecting section (D3-D4) wherein wires run together is 5 mm in length and then attached to Coupling section (D4-D5) or handle further.

This design is very useful at the start of root canal treatment for orifice widening.

### Advantages of the Present Invention are as Follows:

a. Thin flexible wires can easily glide through the thin variable size and shape of the root canal.
b. Thin wires are more resistant to cyclic failure.
c. Absence of any cutting edge will prevent the torsional failure.
d. Basket design will bring the vortex ring type fluid movement in irrigation solution. This will help to cleanse the surface of organic and necrotic debris. It will bring the best result of tissue dissolving property of sodium hypochlorite solution used during the working of file.
e. Thin wires will have minimum cutting action on the dentine. This prevents the excessive thinning of root canal wall that can fracture.
f. Increase the success rate of Root canal treatment
g. Basket design insures the easy removal of complete file, if one of the wire breaks

We claim:

1. A rotary endodontic file in basket form for cleaning and extirpating the walls of a root canal comprising;
i. a tip (D0);
ii. an Edge portion (D0-D1) which act as a single point of contact of wires at lower end of said rotary endodontic file;
iii. a Working section (D1-D3) comprising atleast two thin, flexible wires;
iv. a Connecting section (D3-D4) for connecting said Working section (D1-D3) to Coupling section (D4-D5) and
v. a Coupling section (D4-D5) acting as a handle for said endodontic file for holding the device during root canal treatment.

2. The rotary endodontic file as claimed in claim 1; wherein; the diameter of said edge portion (D0-D1) is in the range of 0.05 mm to 0.8 mm and length is in the range of 0.5 mm to 7 mm.

3. The rotary endodontic file as claimed in claim 1; wherein; said working section (D1-D3) further comprising atleast two thin, tapered or non-tapered flexible wires with thickness ranging from 0.02 mm to 0.4 mm, arranged in such a manner that they form atleast one basket shape.

4. The rotary endodontic file as claim 1; wherein; the working section (D1-D3) has length in range of 12 mm to 21 mm and width raging from 0.4 mm to 6 mm.

5. The rotary endodontic file as claim 1; wherein; the working section (D1-D3) has the flaring angle with respect to long axis at D1 ranges from 1 degree to 24 degree.

6. The rotary endodontic file as claimed in claim 1; wherein; the diameter of said connecting section (D3-D4) is in the range of 0.2 mm to 1 mm and length in the range of 5 mm to 15 mm.

7. The rotary endodontic file as claimed in claim 1; wherein; said coupling section (D4-D5) acts as a handle for holding said rotary endodontic file having diameter in range of 2 mm to 5 mm and length in the range of 7 mm to 20 mm respectively.

8. The rotary endodontic file as claimed in claim 1; wherein; said thin, flexible wire material is selected from stainless steel, any steel alloy, high temperature shape memory alloys, any super elastic material such as but not limited to, nitinol, nitinol-niobium alloy, nitinol alloys with zirconium, any nitinol alloy with or without trace elements, tantalum alloys with or without trace elements, hoffnium alloys, titanium, beta-titanium, titanium alloys with zirconium, any titanium alloy with or without trace elements, shape memory ceramics, silicones or any other polymer and combinations thereof.
